(19) United States
(12) Patent Application Publication
(10) Pub. No.: US 2010/0125291 A1
(43) Pub. Date: May 20, 2010

(54) DRILL AND FLAVORED FLUID PARTICLES COMBINATION

(76) Inventors: Ioana M. Rizoiu, San Clemente, CA (US); Federico Pignatelli, Beverly Hills, CA (US)

Correspondence Address: STOUT, UXA, BUYAN & MULLINS LLP, 4 VENTURE, SUITE 300, IRVINE, CA, 92618, US

(21) Appl. No.: 12/693,370

(22) Filed: Jan. 25, 2010

Related U.S. Application Data
(63) Continuation of application No. 12/336,528, filed on Dec. 16, 2008.
Application No. 12/336,528 is a continuation-in-part of application No. 11/711,945, filed on Feb. 27, 2007.
Application No. 11/711,945 is a continuation of application No. 10/435,325, filed on May 9, 2003, now Pat. No. 7,320,594.
Application No. 10/435,325 is a division of application No. 09/997,550, filed on Nov. 27, 2001, now Pat. No. 6,561,803.
Application No. 09/997,550 is a continuation of application No. 09/256,697, filed on Feb. 24, 1999, now Pat. No. 6,350,123.
Application No. 09/256,697 is a continuation-in-part of application No. 08/985,513, filed on Dec. 5, 1997, now abandoned.
Application No. 10/435,325 is a continuation-in-part of application No. 08/995,241, filed on Dec. 17, 1997, now abandoned.
(60) Provisional application No. 61/014,047, filed on Dec. 16, 2007.

(51) Int. Cl. A61B 17/32; A61B 17/3203

(52) U.S. Cl. 606/167; 433/88

(57) ABSTRACT

A medical apparatus is constructed to have a proximal end and a distal end, with the distal end including a source of mechanical tissue disruption that is configured to treat or ablate a target surface. The source of mechanical tissue disruption includes a tissue-disrupting distal end, and the medical apparatus further includes a flavored particle output for directing flavored particles in a direction toward the tissue-disrupting distal end. Another feature of the present invention includes a medical apparatus having a proximal end and a distal end. The distal end of the medical apparatus includes a source of tissue displacement that is configured to move or displace a portion of the target surface, and that is formed to have a tissue-displacing distal end. The medical apparatus can be configured to have a flavored particle output for directing flavored particles in a direction toward the tissue-displacing distal end.

### CROSS-REFERENCE TO RELATED APPLICATIONS

This application is a continuation of U.S. application Ser. No. 12/336,528 (Att. Docket BI9002CNCPCNCIP), filed Dec. 16, 2008, which claims the benefit of U.S. Provisional Application No. 61/014,047 (Att. Docket BI9002CNCPCNCIPPR), filed Dec. 16, 2007, and which is a continuation-in-part of U.S. application Ser. No. 11/711,945 (Att. Docket BI9002CNCPCN2), filed Feb. 27, 2007, which is a continuation of U.S. application Ser. No. 10/435,325 (Att. Docket BI9002CNCPCNDIV), filed May 9, 2003, issued as U.S. Pat. No. 7,320,594, the entire contents of all which are hereby incorporated by reference. U.S. application Ser. No. 10/435,325 is a divisional of U.S. application Ser. No. 09/997,550 (Att. Docket BI9002CNCPCN), filed Nov. 27, 2001, issued as U.S. Pat. No. 6,561,803, which is a continuation of U.S. application Ser. No. 09/256,697 (Att. Docket BI9002CONCIP), filed Feb. 24, 1999, issued as U.S. Pat. No. 6,350,123, which is a continuation-in-part of U.S. application Ser. No. 08/985,513 (Att. Docket BI9001CON), filed Dec. 5, 1997, now abandoned, the entire contents of all which are hereby incorporated by reference. U.S. application Ser. No. 10/435,325 is also a continuation-in-part of U.S. application Ser. No. 08/995,241 (Att. Docket BI9002CON), filed Dec. 17, 1997, now abandoned, the entire contents of which are hereby incorporated by reference.

### BACKGROUND OF THE INVENTION

1. Field of the Invention

The present invention relates generally to medical drilling techniques and, more particularly, to a system for introducing conditioned fluids into the drilling techniques.

2. Description of Related Art

Water is generally used in a variety of cutting operations in order to cool the target surface. Additionally, water is used in mechanical drilling operations for cooling the target surface and removing cut or drilled materials therefrom. Many prior art cutting or drilling systems use a combination of air and water, commonly combined to form a light mist, for cooling a target surface and/or removing cut materials from the target surface. The use of water in these prior art systems has been somewhat successful for the limited purposes of cooling a target surface or removing debris therefrom. These prior art uses of water in cutting and drilling operations, however, have not allowed for versatility, outside of the two functions of cooling and removing debris.

### SUMMARY OF THE INVENTION

According to an aspect of the present invention, a handpiece is constructed to have a proximal end and a distal end, with the distal end including a source of mechanical tissue disruption that is configured to treat or ablate a target surface. The source of mechanical tissue disruption includes a tissue-disrupting distal end, and the handpiece further includes a flavored particle output for directing flavored particles in a direction toward the tissue-disrupting distal end. Another feature of the present invention includes a handpiece having a proximal end and a distal end. The distal end of the handpiece includes a source of tissue displacement that is configured to move or displace a portion of the target surface, and that is formed to have a tissue-displacing distal end. The handpiece, furthermore, can be configured to have a flavored particle output for directing flavored particles in a direction toward the tissue-displacing distal end. The present invention, together with additional features and advantages thereof, may best be understood by reference to the following description taken in connection with the accompanying illustrative drawings.

While the apparatus and method have or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described or referenced herein are included within the scope of the present invention provided that the features included in any such combination are not mutually inconsistent as will be apparent from the context, this specification, and the knowledge of one skilled in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and additional disclosure in claims format that follow.

### BRIEF DESCRIPTION OF THE FIGURE

FIG. 1 illustrates a drill assembly provided with a source of flavored fluid particles according to an embodiment of the present invention.

### DETAILED DESCRIPTION OF THE PRESENT INVENTION

Reference will now be made to certain embodiments (e.g., certain described and/or incorporated embodiments) of the invention, partial examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain embodiments, it is to be understood that these embodiments are presented by way of example and not by way of limitation. The intent of this disclosure, while discussing exemplary embodiments, is that the following detailed description be construed to cover all modifications, alternatives, and equivalents of the embodiments as may fall within the spirit and scope of the invention as defined by the additional disclosure in claims format. It is to be understood and appreciated that the process steps and structures described herein do not cover a complete architecture or process, and only so much of the commonly practiced features and steps are included herein as are necessary to provide an understanding of the present invention.

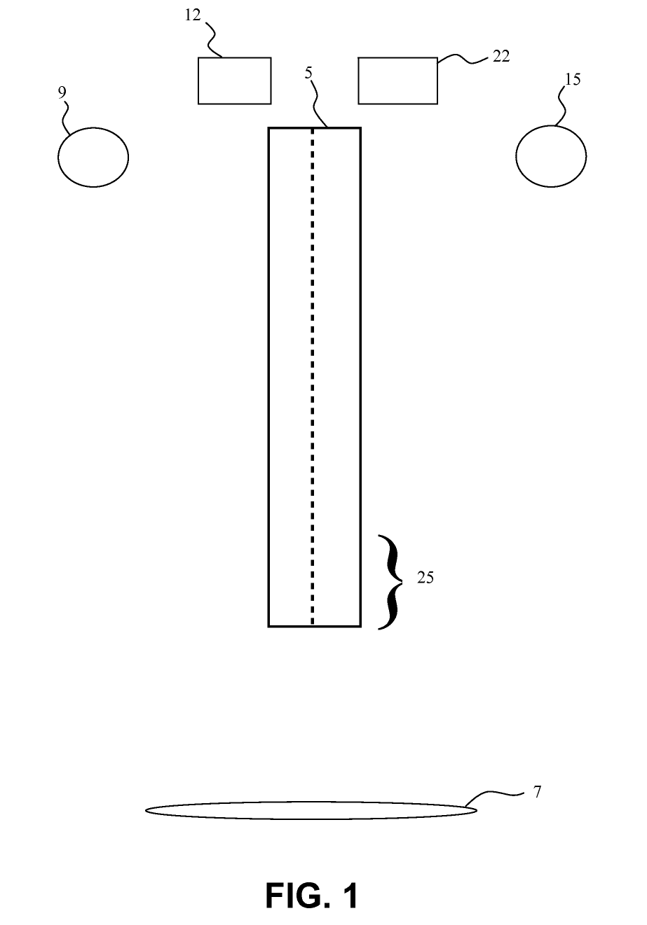

As depicted in the attached figures, in connection with certain aspects of the present invention, a drill assembly is provided with a source of flavored fluid particles. As depicted in the attached figures, in connection with other aspects of the present invention, a drill assembly is provided with a source of flavored fluid particles and also with a source of electromagnetic energy. In particular, FIG. 1 illustrates a drill assembly that is provided with a source of fluid particles according to embodiments of the present invention. The drill assembly can comprise one or more drills 5, one or more first outputs 9, one or more second outputs 12, one or more third outputs 15 and one or more fourth outputs 22. Each, or any number of, the outputs can be configured and oriented for directing one or more of (a) any electromagnetic energy described or referenced herein, (b) a liquid, (c) a gas, and (d) a combination of gas and liquid, toward the distal region 25. The elements 5, the fluid, and electromagnetic energy can comprise any characteristics described and referenced herein (e.g., drill can be transparent or have a shaped region 25 for ablating, etc.). In one implementation, a first handpiece comprises a drill 5, output 9 and output 12, and a second handpiece comprises a second drill 5 (symbolized as the right part of the phantom line, although the drills 5 and second drill 5 would of course not touch, being part of different handpieces), a second output 15 and a second output 22.

According to one broad aspect, rather than or in addition to a drill assembly, a source of mechanical tissue disruption is provided with the same or an additional/alternative source of flavored fluid particles. The source of mechanical tissue disruption can be configured to treat or ablate a target surface. Furthermore, the source of mechanical tissue disruption can comprise a tissue-disrupting distal end that performs one or more of contacting the target surface and effectuating ablation of a portion of the target surface.

According to another broad aspect, rather than or in addition to the drill assembly, a source of tissue displacement is provided with a source of flavored fluid particles. The source of tissue displacement can be configured to effectuate one or more of contacting the target surface and moving or displacing a portion of the target surface. Furthermore, the source of tissue displacement can comprise a tissue-displacing distal end that contacts the target surface and moves or displaces a portion of the target surface.

The source of flavored fluid particles may also be provided with, in certain implementations, a source of electromagnetic energy, which may comprise laser energy and/or visible light and may operate to provide or promote one or more of desterilization, bacterial reduction, biostimulation (e.g., low-level light therapy), coagulation, remodeling, caries detection or treatment, and illumination (e.g., with visible light).

The handpiece may comprise a dental handpiece with a dental tip (e.g., comprising a dental drill).

In one implementation, a drill may be configured with a source of flavored fluid particles and further (a) may be configured with an electromagnetic energy source of ablation, and/or (b) the same drill or an alternative drill may be configured with an electromagnetic energy source of illumination, and/or (c) the same or an alternative drill may be configured with an electromagnetic energy source of tissue disruption, and/or (d) the same or an alternative drill may be configured with an electromagnetic energy source of biostimulation. The drill may comprise, according to one implementation, a dental drill for cutting oral tissue.

The target surface may comprise, for example, one or more of tooth tissue, bone, cartilage and soft tissue such as skin.

According to certain aspects of the present invention, an energy output can be configured to emit electromagnetic energy, which can comprise one or more of hard-tissue ablating electromagnetic energy, low-level light therapy (LLLT) electromagnetic energy, tissue-biostimulation electromagnetic energy, visible electromagnetic energy, coherent light, one or more of a wavelength within a range from about 2.69 to about 2.80 microns and a wavelength of about 2.94 microns, and electromagnetic energy generated by one or more of an Er:YAG laser, an Er:YSGG laser, an Er, a Cr:YSGG laser and a CTE:YAG laser.

The assembly can comprise a fluid port disposed at a first location of the distal end of the handpiece for emitting the flavored fluid particles, and another structure (e.g., port that can accommodate a powered rotating shaft for, as an example, a drill) at a second location of the distal end of the handpiece can be provided with the tissue-disrupting end or the tissue-displacing distal end.

The assembly can comprise a handpiece. The handpiece can further comprise an electromagnetic energy output configured to emit energy in a vicinity of the distal end of the handpiece.

In one implementation, the electromagnetic energy output can be configured to direct electromagnetic energy in a direction toward the tissue-disrupting or the tissue-displacing distal end. For example, a electromagnetic energy port(s) can be provided at one or more of the first location, the second location, and a third location. The electromagnetic energy port(s) can be the same as or different from the above-mentioned fluid port at the first location and/or the above-mentioned structure at the second location, and the electromagnetic energy port(s) can be provided with or without the flavored particle output and/or the structure being located at the respective ports or port vicinities.

The flavored fluid can be a flavored liquid. In certain implementations, the liquid is or comprises flavored water.

According to other implementations, the apparatus comprises a fluid output that is configured to emit fluid, which may be the flavored fluid particles and/or other fluid, in a vicinity of the distal end of the apparatus, wherein: the fluid output comprises an atomizer configured to place atomized fluid particles into a volume above the target surface; and the energy output is configured to impart relatively large amounts of energy into the atomized fluid particles in the volume above the target surface to thereby expand the atomized fluid particles and impart disruptive forces onto the target surface. The other fluid may be or may comprise conditioned fluid or conditioned fluid particles, as described or referenced in any of the below-cited documents.

In certain implementations, the tissue-disrupting or the tissue-displacing distal end (e.g., drill end) may be able to ablate or remove the target surface (e.g., hard tissue) better than a laser. According to other implementations, the tissue-disrupting or the tissue-displacing distal end (e.g., a drill end or a side-firing drill end) may be able to access, for example, sidewalls within a lumen, channel, pocket, etc. of the target and/or remove the target surface (e.g., hard tissue) better than a laser.

In still other implementations, the tissue-disrupting or the tissue-displacing distal end (e.g., a drill end or a side-firing drill end) may comprise a clear material and/or a material that is transparent to one or more wavelengths of the electromagnetic energy. For instance, the tissue-disrupting or the tissue-displacing distal end may comprise one or more of sapphire, diamond, and a ceramic material. The attached figures elucidate exemplary tissue-disrupting or tissue-displacing distal ends.

Corresponding or related structure and methods described in the following patents assigned to Biolase Technology, Inc., are incorporated herein by reference in their entireties, wherein such incorporation includes corresponding or related structure (and modifications thereof) in the following patents which may be, in whole or in part, (i) operable with, (ii) modified by one skilled in the art to be operable with, and/or (iii) implemented/used with or in combination with, any part(s) of the present invention according to this disclosure, that of the patents or below applications, and the knowledge and judgment of one skilled in the art:

Such patents include, but are not limited to, U.S. Pat. No. 7,356,208 entitled Fiber detector apparatus and related methods; U.S. Pat. No. 7,320,594 entitled Fluid and laser system; U.S. Pat. No. 7,303,397 entitled Caries detection using timing differentials between excitation and return pulses; U.S. Pat. No. 7,292,759 entitled Contra-angle rotating handpiece having tactile-feedback tip ferrule; U.S. Pat. No. 7,290,940 entitled Fiber tip detector apparatus and related methods; U.S. Pat. No. 7,288,086 entitled High-efficiency, side-pumped diode laser system; U.S. Pat. No. 7,270,657 entitled Radiation emitting apparatus with spatially controllable output energy distributions; U.S. Pat. No. 7,261,558 entitled Electromagnetic radiation emitting toothbrush and dentifrice system; U.S. Pat. No. 7,194,180 entitled Fiber detector apparatus and related methods; U.S. Pat. No. 7,187,822 entitled Fiber tip fluid output device; U.S. Pat. No. 7,144,249 entitled Device for dental care and whitening; U.S. Pat. No. 7,108,693 entitled Electromagnetic energy distributions for electromagnetically induced mechanical cutting; U.S. Pat. No. 7,068,912 entitled Fiber detector apparatus and related methods; U.S. Pat. No. 6,942,658 entitled Radiation emitting apparatus with spatially controllable output energy distributions; U.S. Pat. No. 6,829,427 entitled Fiber detector apparatus and related methods; U.S. Pat. No. 6,821,272 entitled Electromagnetic energy distributions for electromagnetically induced cutting; U.S. Pat. No. 6,744,790 entitled Device for reduction of thermal lensing; U.S. Pat. No. 6,669,685 entitled Tissue remover and method; U.S. Pat. No. 6,616,451 entitled Electromagnetic radiation emitting toothbrush and dentifrice system; U.S. Pat. No. 6,616,447 entitled Device for dental care and whitening; U.S. Pat. No. 6,610,053 entitled Methods of using atomized particles for electromagnetically induced cutting; U.S. Pat. No. 6,567,582 entitled Fiber tip fluid output device; U.S. Pat. No. 6,561,803 entitled Fluid conditioning system; U.S. Pat. No. 6,544,256 entitled Electromagnetically induced cutting with atomized fluid particles for dermatological applications; U.S. Pat. No. 6,533,775 entitled Light-activated hair treatment and removal device; U.S. Pat. No. 6,389,193 entitled Rotating handpiece; U.S. Pat. No. 6,350,123 entitled Fluid conditioning system; U.S. Pat. No. 6,288,499 entitled Electromagnetic energy distributions for electromagnetically induced mechanical cutting; U.S. Pat. No. 6,254,597 entitled Tissue remover and method; U.S. Pat. No. 6,231,567 entitled Material remover and method; U.S. Pat. No. 6,086,367 entitled Dental and medical procedures employing laser radiation; U.S. Pat. No. 5,968,037 entitled User programmable combination of atomized particles for electromagnetically induced cutting; U.S. Pat. No. 5,785,521 entitled Fluid conditioning system; and U.S. Pat. No. 5,741,247 entitled Atomized fluid particles for electromagnetically induced cutting.

Also, the above disclosure and referenced items, and that described on the referenced pages, are intended to be operable or modifiable to be operable, in whole or in part, with corresponding or related structure and methods, in whole or in part, described in the following published applications and items referenced therein, which applications are listed as follows: App. Pub. 20080125677 entitled Methods for treating hyperopia and presbyopia via laser tunneling; App. Pub. 20080125676 entitled Methods for treating hyperopia and presbyopia via laser tunneling; App. Pub. 20080097418 entitled Methods for treating eye conditions; App. Pub. 20080097417 entitled Methods for treating eye conditions; App. Pub. 20080097416 entitled Methods for treating eye conditions; App. Pub. 20080070185 entitled Caries detection using timing differentials between excitation and return pulses; App. Pub. 20080065057 entitled High-efficiency, side-pumped diode laser system; App. Pub. 20080065055 entitled Methods for treating eye conditions; App. Pub. 20080065054 entitled Methods for treating hyperopia and presbyopia via laser tunneling; App. Pub. 20080065053 entitled Methods for treating eye conditions; App. Pub. 20080033411 entitled High efficiency electromagnetic laser energy cutting device; App. Pub. 20080033409 entitled Methods for treating eye conditions; App. Pub. 20080033407 entitled Methods for treating eye conditions; App. Pub. 20080025675 entitled Fiber tip detector apparatus and related methods; App. Pub. 20080025672 entitled Contra-angle rotating handpiece having tactile-feedback tip ferrule; App. Pub. 20080025671 entitled Contra-angle rotating handpiece having tactile-feedback tip ferrule; App. Pub. 20070298369 entitled Electromagnetic radiation emitting toothbrush and dentifrice system; App. Pub. 20070263975 entitled Modified-output fiber optic tips; App. Pub. 20070258693 entitled Fiber detector apparatus and related methods; App. Pub. 20070208404 entitled Tissue treatment device and method; App. Pub. 20070208328 entitled Contra-angel rotating handpiece having tactile-feedback tip ferrule; App. Pub. 20070190482 entitled Fluid conditioning system; App. Pub. 20070184402 entitled Caries detection using real-time imaging and multiple excitation frequencies; App. Pub. 20070104419 entitled Fiber tip fluid output device; App. Pub. 20070060917 entitled High-efficiency, side-pumped diode laser system; App. Pub. 20070059660 entitled Device for dental care and whitening; App. Pub. 20070054236 entitled Device for dental care and whitening; App. Pub. 20070054235 entitled Device for dental care and whitening; App. Pub. 20070054233 entitled Device for dental care and whitening; App. Pub. 20070042315 entitled Visual feedback implements for electromagnetic energy output devices; App. Pub. 20070014517 entitled Electromagnetic energy emitting device with increased spot size; App. Pub. 20070014322 entitled Electromagnetic energy distributions for electromagnetically induced mechanical cutting; App. Pub. 20070009856 entitled Device having activated textured surfaces for treating oral tissue; App. Pub. 20070003604 entitled Tissue coverings bearing customized tissue images; App. Pub. 20060281042 entitled Electromagnetic radiation emitting toothbrush and dentifrice system; App. Pub. 20060275016 entitled Contra-angle rotating handpiece having tactile-feedback tip ferrule; App. Pub. 20060241574 entitled Electromagnetic energy distributions for electromagnetically induced disruptive cutting; App. Pub. 20060240381 entitled Fluid conditioning system; App. Pub. 20060210228 entitled Fiber detector apparatus and related methods; App. Pub. 20060204203 entitled Radiation emitting apparatus with spatially controllable output energy distributions; App. Pub. 20060142743 entitled Medical laser having controlled-temperature and sterilized fluid output; App. Pub. 20060099548 entitled Caries detection using timing differentials between excitation and return pulses; App. Pub. 20060043903 entitled Electromagnetic energy distributions for electromagnetically induced mechanical cutting; App. Pub. 20050283143 entitled Tissue remover and method; App. Pub. 20050281887 entitled Fluid conditioning system; App. Pub. 20050281530 entitled Modified-output fiber optic tips; App. Pub. 20040106082 entitled Device for dental care and whitening; App. Pub. 20040092925 entitled Methods of using atomized particles for electromagnetically induced cutting; App. Pub. 20040091834 entitled Electromagnetic radiation emitting toothbrush and dentifrice system; App. Pub. 20040068256 entitled Tissue remover and method; App. Pub. 20030228094 entitled Fiber tip fluid output device; App. Pub. 20020149324 entitled Electromagnetic energy distributions for electromagnetically induced mechanical cutting; and App. Pub. 20020014855 entitled entitled Electromagnetic energy distributions for electromagnetically induced mechanical cutting.

All of the contents of the preceding published applications are incorporated herein by reference in their entireties.

The above-described embodiments have been provided by way of example, and the present invention is not limited to these examples. Multiple variations and modifications to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the source of mechanical tissue disruption, source of mechanical tissue disruption or sources of tissue displacement (e.g., drills), flavored particle outputs (e.g., comprising an atomizer), energy outputs (e.g., lasers), and other components including handpiece apparatus, and any particulars or features thereof, or other features, including method steps and techniques, may be used with any other structure and process described or referenced herein, in whole or in part, in any combination or permutation as a non-equivalent, separate, non-interchangeable aspect of this application. Accordingly, the present invention is not intended to be limited by the disclosed embodiments, but is to be defined by such embodiments and by reference to the following claims.

1-19. (canceled)

20. An apparatus for implementing a medical procedure, comprising:
a user control for inputting a user-specified combination of atomized fluid particles, the user-specified combination of atomized fluid particles corresponding to a user-specified average size, spatial distribution, and velocity of atomized fluid particles;
a fluid output for supplying fluid to a first vicinity relative to the fluid output, the fluid output comprising a biologically compatible fluid, being constructed to route the biologically compatible fluid into the first vicinity simultaneously with the supplying of electromagnetic energy to a second vicinity, and further comprising an atomizer responsive to the user control for generating the combination of biologically compatible atomized fluid particles and for placing the combination of biologically compatible atomized fluid particles into said first vicinity; and
an electromagnetic energy source for supplying the electromagnetic energy to the second vicinity for at least partial absorption by said fluid, wherein the first vicinity and the second vicinity intersect in a volume relative to the fluid output which is positioned in use above a target surface, wherein said electromagnetic energy source comprises a specifically configured electromagnetic energy source that is arranged and constructed to route electromagnetic energy through a fiber optic and to emit a peak concentration of the electromagnetic energy into the second vicinity simultaneously with the routing of the combination of biologically compatible atomized fluid particles into the first vicinity;
wherein said fluid output is configured to route the combination of biologically compatible atomized fluid particles and distribute them into the volume in such a way as, when said apparatus is positioned in use such that said volume is above said target surface, to supply electromagnetic energy of a wavelength which is substantially absorbed by said combination of biologically compatible atomized fluid particles and to focus or place a peak concentration of said electromagnetic energy into said volume so as to be substantially absorbed by at least a portion of said combination of biologically compatible atomized fluid particles to cause the portion of the combination of biologically compatible atomized fluid particles to expand, whereby during use disruptive mechanical forces are imparted to said target surface.

21. An apparatus for implementing a medical procedure, comprising:
a fluid output for supplying fluid to a first vicinity relative to the fluid output; and
an electromagnetic energy source for supplying electromagnetic energy to a second vicinity for at least partial absorption by said fluid;
characterized in that:
the first vicinity and the second vicinity intersect in a volume relative to the fluid output which is positioned in use above a target surface;
said apparatus comprises a user control for inputting a user-specified combination of atomized fluid particles, the user-specified combination of atomized fluid particles corresponding to a user-specified average size, spatial distribution, and velocity of atomized fluid particles;
said fluid output comprises a biologically compatible fluid, is constructed to route the biologically compatible fluid into the first vicinity simultaneously with the supplying of the electromagnetic energy to the second vicinity, and further comprises an atomizer responsive to the user control for generating the combination of biologically compatible atomized fluid particles and for placing the combination of biologically compatible atomized fluid particles into said volume;
said electromagnetic energy source comprises a specifically configured electromagnetic energy source that is arranged and constructed to route electromagnetic energy through a fiber optic and to emit a peak concentration of the electromagnetic energy into the second vicinity simultaneously with the routing of the combination of biologically compatible atomized fluid particles; and
said fluid router is configured to route the combination of biologically compatible atomized fluid particles and distribute them into the volume in such a way as, when said apparatus is positioned in use such that said volume is above said target surface, to supply electromagnetic energy of a wavelength which is substantially absorbed by said combination of biologically compatible atomized fluid particles and to focus or place a peak concentration of said electromagnetic energy into said volume so as to be substantially absorbed by at least a portion of said combination of biologically compatible atomized fluid particles to cause the portion of the combination of biologically compatible atomized fluid particles to expand, whereby during use disruptive mechanical forces are imparted to said target surface.

22. The apparatus as set forth in claim 21, wherein the biologically compatible atomized fluid particles contact the fiberoptic.

23. The apparatus as set forth in claim 22, wherein the user control comprises an input device for enabling inputting by a user corresponding to one of a high resolution cut and a low resolution cut, and to one of a deep-penetration cut and a shallow-penetration cut.

24. The apparatus as set forth in claim 23, wherein the atomizer is responsive to the input device and is configured to generate a combination of atomized fluid particles comprising relatively small fluid particles in response to a user input specifying a high resolution cut or to generate a combination of atomized fluid particles comprising larger fluid particles in response to a user input specifying a low resolution cut.

25. The apparatus as set forth in claim 23, wherein the atomizer is responsive to the input device and is configured to generate a combination of atomized fluid particles comprising a relatively low-density distribution of fluid particles in response to a user input specifying a deep-penetration cut and to generate a combination of atomized fluid particles which comprises a higher-density distribution of fluid particles in response to a user input specifying a shallow-penetration cut.

26. The apparatus as set forth in claim 23, wherein the input device comprises a single input for controlling cutting efficiency.

27. The apparatus as set forth in claim 26, wherein the input device generates a relatively low-density distribution of relatively small fluid particles when the single input specifies a high cutting efficiency.

28. The apparatus as set forth in claim 22, wherein the user control comprises an input device, and wherein the input device comprises a single input for controlling cutting efficiency.

29. The apparatus as set forth in claim 28, wherein the user input device generates a relatively high-density distribution of relatively large fluid particles when the single input specifies a low cutting efficiency.

30. The apparatus as set forth in claim 29, wherein the atomizer generates a combination of atomized fluid particles comprising relatively large fluid particles in response to the user input specifying a low resolution cut.

31. The apparatus as set forth in claim 22, the apparatus having a configuration to select an upstream fluid pressure in response at least in part to a user input corresponding to penetration level, and to cause the atomizer to apply the upstream fluid pressure to a fluid spray nozzle thereof, to thereby generate the user-specified combination of biologically compatible atomized fluid particles.

32. The apparatus as set forth in claim 31, wherein the atomizer is connected and configured to generate a combination of biologically compatible atomized fluid particles comprising relatively small fluid particles in response to the user input specifying a high resolution cut.

33. The apparatus as set forth in claim 21, wherein the electromagnetic energy source comprises one of a wavelength within a range from about 2.69 to about 2.80 microns and a wavelength of about 2.94 microns.

34. The apparatus as set forth in claim 21, wherein the electromagnetic energy source comprises one of an Er:YAG, an Er:YSGG, an Er, a Cr:YSGG and a CTE:YAG laser.

35. The apparatus as set forth in claim 21, wherein the target surface comprises one of tooth, bone, cartilage and soft tissue, and the fluid particles comprise water.

36. The apparatus as set forth in claim 21, wherein the electromagnetic energy source is configured to impart relatively large amounts of energy into the atomized fluid particles in the volume above the target surface to thereby expand the atomized fluid particles and impart the disruptive mechanical forces to the target surface.

37. The apparatus as set forth in claim 21, wherein the electromagnetic energy source comprises a diode.

38. The apparatus as set forth in claim 22, wherein the electromagnetic energy source comprises a diode.

39. The apparatus as set forth in claim 38, wherein the fluid comprises water and the target surface comprises one of tooth, bone, cartilage and soft tissue.

40. The apparatus as set forth in claim 21, wherein the fluid particles comprise water.
